(19) United States
(12) Patent Application Publication
(10) Pub. No.: US 2011/0230702 A1
(43) Pub. Date: Sep. 22, 2011

(54) Device, System, And Method For Treating Sleep Apnea

(76) Inventor: Kirk Honour, Shorewood, MN (US)

(21) Appl. No.: 13/042,583

(22) Filed: Mar. 8, 2011

Related U.S. Application Data
(60) Provisional application No. 61/314,294, filed on Mar. 16, 2010.
(60) Provisional application No. 61/361,519, filed on Jul. 5, 2010.
(60) Provisional application No. 61/326,800, filed on Apr. 22, 2010.

(51) Int. Cl. A61N 1/36; A61N 2/00; A61N 2/02

(52) U.S. Cl. 600/13; 607/42; 600/15

(57) ABSTRACT

In an embodiment, a mask is used to position electrodes on a user so current traveling between the electrodes can stimulate nerves that control the geometry of the mask user's airway (e.g., pharynx, neck, throat, mouth, trachea, and the like). In an embodiment, a collar is used to position electrodes on a user so current travelling between the electrodes can stimulate nerves that control the geometry of the collar user's airway. Any of the above current may help treat apnea via direct or indirect stimulation of muscles or nerves.

This application claims priority to U.S. Provisional Patent Application No. 61/314,294 filed on Mar. 12, 2010 and entitled “Sleep Apnea Treatment System Using Magnetic Stimulation of the Phrenic Nerve”, the content of which is hereby incorporated by reference. This application claims priority to U.S. Provisional Patent Application No. 61/361,519 filed on Apr. 5, 2010 and entitled “Medical Treatment System Using Stimulation of Peripheral Nerves”, the content of which is hereby incorporated by reference. This application claims priority to U.S. Provisional Patent Application No. 61/326,800 filed on Apr. 22, 2010 and entitled “Medical Treatment System Using Magnetic Stimulation of Peripheral Nerves”, the content of which is hereby incorporated by reference.

### BACKGROUND

Forms of sleep apnea include central sleep apnea (CSA), obstructive sleep apnea (OSA), and mixed form sleep apnea that is a combination of CSA and OSA.

CSA includes a group of sleep-related breathing disorders in which respiratory effort is diminished or absent in an intermittent or cyclical fashion. More specifically, in CSA the basic neurological controls for breathing rate malfunction and fail to give the signal to inhale, causing the individual to miss one or more cycles of breathing. During polysomnography (PSG), a central apneic event may include cessation of airflow for 10 seconds or longer without an identifiable respiratory effort.

CSA is often associated with OSA syndromes or may be caused by, for example, an underlying medical condition. Several different entities are grouped under CSA with varying signs, symptoms, and clinical and PSG features. Those that affect adults include primary CSA, Cheyne-Stokes breathing-central sleep apnea (CSBCSA) pattern, high-altitude periodic breathing, CSA due to medical conditions other than Cheyne-Stokes, and CSA due to drug or substance interaction. CSBCSA may be affiliated with patients suffering from heart failure and/or stroke.

OSA may occur because muscle tone for airway muscles relaxes during sleep. More specifically, at throat level the human airway is composed of collapsible walls of soft tissue. Upon loss of muscle tone the muscles collapse into the airway and obstruct breathing during sleep. An obstructive apneic event has a discernible ventilatory effort during the period of airflow cessation. More severe forms of OSA may require treatment to prevent low blood oxygen levels, sleep deprivation, mood alterations, memory loss, dementia, and even cardiovascular disease including congestive heart failure and atrial fibrillation.

Individuals with sleep apnea may be unaware (even upon awakening) of having experienced difficulty breathing while asleep. Sleep apnea is usually first recognized as a problem by others witnessing the affected individual during apnea episodes or is suspected because of its effects on the patient. Symptoms may be present for years without identification of the underlying sleep apnea, during which time the sufferer may become conditioned to the daytime fatigue associated with significant levels of sleep disturbance.

### BRIEF DESCRIPTION OF THE DRAWINGS

Features and advantages of the present invention will become apparent from the appended claims, the following detailed description of one or more example embodiments, and the corresponding figures, in which:

FIG. 1 includes a mask in an embodiment of the invention.

FIG. 2 includes a collar in an embodiment of the invention.

FIGS. 3A, B include stimulus vectors in embodiments of the invention.

FIG. 4 includes a vest in an embodiment of the invention.

FIG. 5 includes a mask in an embodiment of the invention.

FIG. 6 includes a system for use with an embodiment of the invention.

### DETAILED DESCRIPTION

In the following description, numerous specific details are set forth but embodiments of the invention may be practiced without these specific details. Well-known circuits, structures and techniques have not been shown in detail to avoid obscuring an understanding of this description. “An embodiment”, “example embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Coupled” and “connected” and their derivatives are not synonyms. “Connected” may indicate elements are in direct physical or electrical contact with each other and “coupled” may indicate elements co-operate or interact with each other, but they may or may not be in direct physical or electrical contact.

In an embodiment of the invention, a mask is used to position electrodes on a user so current traveling between the electrodes can stimulate nerves that control the geometry of the mask user's airway (e.g., pharynx, neck, throat, mouth, trachea, and the like).

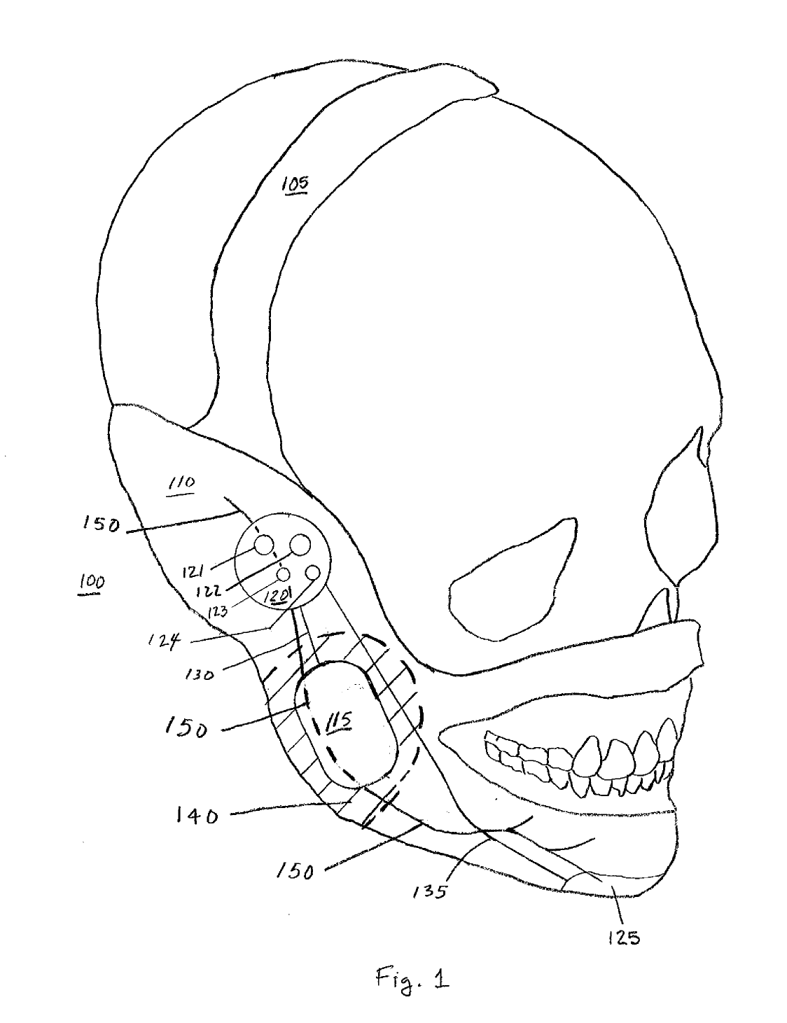

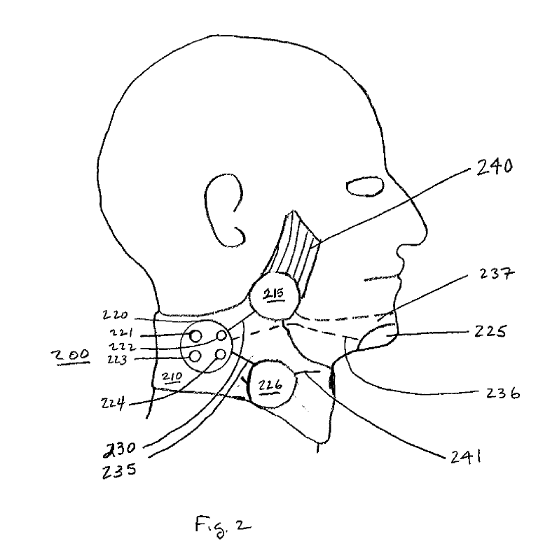

FIG. 1 includes a mask in an embodiment of the invention. Mask 100 includes first portion 110 that includes electrode 115, electrode 125, and module 120. Elements 115, 125, and 120 may be coupled to one another via interconnects (e.g., wires) 130, 135. Module 120 may include power source 121 (e.g., rechargeable battery) and/or controller 122. Headstrap 105 is included in some embodiments. Also, power source 121 may not necessarily include a battery but may instead couple to auxiliary power via, for example, an adaptor coupled to a power source (e.g., 110 volt power). Further, user interface 123 (e.g., liquid crystal display, graphical user interface) may display various modes (e.g., different pacing regimes, summary of sensed events, battery life, current amplitude, current ramping, on/off, and the like) that can be advanced through using input (e.g., key) 124. Electrode 115 may be the anode and electrode 125 may be the cathode. The resultant invoked nerve and/or muscle response may occur near the cathode under the chin. However, in other embodiments electrode 115 may be the cathode and electrode 125 may be the anode. The resultant invoked nerve and/or muscle response may occur near the cathode at or near the temporomandibular joint (TMJ).

In an embodiment, mask 100 provides bilateral stimulation to the user (but in other embodiments may provide for only unilateral stimulation). For example, FIG. 1 depicts electrode 115 near the patient's right TMJ along with electrode 125 near (e.g., on or adjacent) the chin. Specifically, in one embodiment electrode 115 is placed over the upper masticatory muscles, below the cheek bone, and lateral to the eye sockets. In other embodiments, electrode 115 is lateral from (and inline to) the upper palate and directly above the dorsal angle of the lower mandible. While not shown in FIG. 1, another electrode may be located near the patient's left TMJ. The left TMJ electrode could provide stimulation current to electrode 125 or to another electrode located near the chin (i.e., include both and left electrode pairs to include at least four electrodes). Electrode 125 may be directly beneath the chin or, for example, to the left or right side of the under chin area (e.g., if two pairs of electrodes are used the chin electrodes may be slightly offset respectively to the left and right under chin area). The muscles above electrode 125 may include the digastric, mylohyoid, or genioglossus muscles.

Also, the left TMJ electrode may receive power from module 120 or from another module located on the left side of mask 100. Also, controller 122 could be used to drive stimulus (e.g., different drive drains) via the left TMJ or another controller could do so. As a result, stimulus to both the left and right TMJs may provide simultaneous stimulation to left and right nerve bundles located in the jaw and neck. Such nerves may control the muscles surrounding the airway. By stimulating these nerves the “airway muscles” are activated and the airway is kept open.

In various embodiments, the stimulus along the left and right sides of the mask may be equal. However, in other embodiments the stimulus along the left and right sides of the mask may be unequal so a user can program different current levels to account for different needs. For example, target nerves may not be located symmetrically on the user. A left branch of a target nerve may be located further away from the left TMJ electrode than the right branch is located from the right TMJ electrode. As such, current between the left TMJ electrode and a chin electrode may need to be adjusted (e.g., increased). The unequal stimulation may be performed using multiple controllers or a single controller with capacity to perform separate pacing regimes for left and right stimulation.

In an embodiment, mask 100 may stimulate (e.g., continuously or periodically) target nerves and/or muscles based on a programmed pulsing schedule delivered via, for example, controller 122. Target nerves for electrode 115 include peripheral nerves in the head and neck. In an embodiment, the stimulus vector between electrodes 115 and 125 effectively stimulates the hypoglossal nerve (HGN) 150. Other embodiments may focus on stimulating the masticator/masseteric nerve (MN). Still other embodiments stimulate the HGN and MN as well as combinations of other nerves.

The MN includes a smaller root of the trigeminal nerve, composed of fibers originating from the trigeminal motor nucleus and emerging from the pons medial to the much larger sensory root, to join the mandibular nerve. The MN carries motor and proprioceptive fibers to the muscles derived from the first bronchial (mandibular) arch, including the four muscles of mastication, plus the mylohyoid, anterior belly of the digastric, and the tensores tympani and veli palati. Thus, target muscles for electrode 115 stimulation include the immediately aforementioned muscles, masseter muscle 140, and/or pharyngeal airway muscles such as the geniohyoid, genioglossus, styloglossus, and hypoglossus muscles.

Stimulation between or based on electrodes 115 and 125 may directly or indirectly stimulate the HGN 150 by activating the jaw closing muscle sequence. Activating the masseter muscle and MN may cause afferent nerve impulses that are routed to the brain and processed by central motor programs that are located in the medulla and pons of the brainstem and that transform afferent and efferent signals into rhythmic and patterned behaviors. The efferent control pattern may steady the tongue when biting, swallowing and breathing. Thus, by directly or indirectly controlling the MN the HGN 150 can be activated and retrusion of the tongue suppressed.

Stimulation between or based on electrodes 115 and 125 may directly or indirectly stimulate the anterior belly of the digastric muscle. The anterior belly of the digastric muscle is located under the chin and connects the hypoid bone to the area of the lower mandible that forms the chin. When the anterior belly of the digastric muscle is stimulated the muscle pulls the tongue forward and up when the hypoid bone is not stabilized, otherwise stimulation of the anterior belly of the digastric opens the jaw.

Stimulation between or based on electrodes 115 and 125 may directly or indirectly stimulate the geniohyoid muscle. The geniohyoid muscle is located under the chin and connects the os hyoideum to the interior area of the lower mandible that forms the chin. When the geniohyoid muscle is stimulated the muscle pulls the tongue forward and up when the hypoid bone is not stabilized, otherwise stimulation of the anterior belly of the digastric opens the jaw.

Stimulation between or based on electrodes 115 and 125 may directly or indirectly stimulate the mylohyoid muscle. The mylohyoid muscle is located under the chin and connects the os hyoideum to the interior area of the lower mandible that forms the chin. When the mylohyoid muscle is stimulated the muscle pulls the tongue forward and up when the hypoid bone is not stabilized, otherwise stimulation of the anterior belly of the digastric opens the jaw.

Stimulation between or based on electrodes 115 and 125 may directly or indirectly stimulate the genioglossus muscle. The genioglossus muscle is located under the chin and connects the lower tongue body to the interior area of the lower mandible that forms the chin. When the genioglossus muscle is stimulated the muscle pulls the tongue forward toward the mandible.

The stimulus vector between electrodes 115 and 125 may take advantage of its proximity to the mandible to supply sufficient current that does not dissipate too readily (which can occur in areas of less bone and more muscle tissue such as the neck). Thus, the stimulus vector can use a minimum amount of power to stimulate nerves and muscles that are relatively “shallow” and or less internal than other nerves and muscles located in, for example, the neck. Such “deep” nerves and muscles in the neck may sometimes require invasive procedures to implant electrodes within the user. The same amount of current applied between electrodes patches 115 and 125 may produce a larger muscle action potential than the same amount of current applied along a vector emanating from a neck-based electrode.

Also, the stimulus vector between electrodes 115 and 125 may affect fewer non-target muscles (which can be an issue when attempting to simulate the HGN 150 using electrodes more focused on the neck). More specifically, because the stimulus vector is more directly applied to key nerves (as opposed to general application to muscle mass to produce indirect nerve stimulation) less current may be needed for desired results and non-target nerves are less likely to be indirectly stimulated based on stimulation of related muscle. For example, stimulating with a patch on the neck may cause inadvertent stimulation of shoulder, neck, and/or back muscles based on misdirected stimulus vectors created by the neck-based electrode.

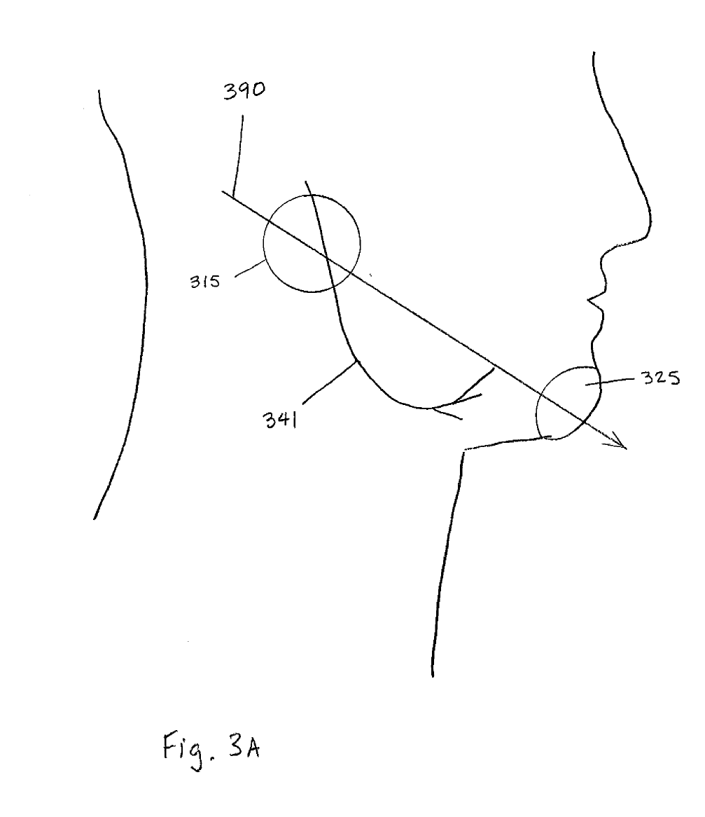

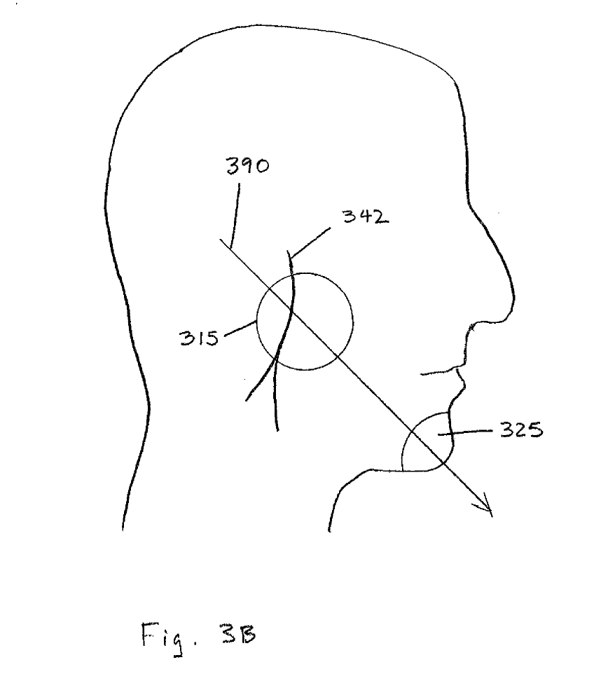

FIG. 3A shows stimulus vector 390 (based on current supplied between electrodes 315, 325) traversing HGN 341. FIG. 3B shows the same stimulus vector 390 (based on current supplied between electrodes 315, 325) traversing MN 342.

Input 124 may, for example, allow a user to increase current levels to provide proper therapy. For instance, increased current levels may be needed if target nerves or muscles are located at relatively longer distances from stimulating electrodes. Also, increasing current levels may accommodate variances in skin conductivity, muscle thickness, fat or adipose tissue thickness, and the like. Also, key 124 (or some other input means) may toggle through various modes that vary in, for example, pulse width duration, pulse drain duration, rest period duration between pulse trains, and the like.

In an embodiment, a drive train with the following characteristics is supplied to the masseter muscle: stimulate every 6 seconds with 2 second pulses. Different modes or stimulus algorithms may be stored in memory included in or coupled to controller 122. A user may toggle, via key 124, to trains that pace every 3, 4, 5, 6, 7, 8, 9, or 10 seconds with pulse widths of 1, 2, 3, 4, 5, 6, 7 seconds. Far more infrequent pacing may occur such as 1, 2, 3, 4, 5, 6, 7, 8 and the like times/night. As noted above, due to efficient placement of stimulus vectors (e.g., 340, 341) over the mandible area (where there is less fat and muscle tissue) embodiments may still stimulate target muscles albeit with relatively lower amounts of current. For example, an embodiment uses less than 5 watts of power for stimulation. Embodiments may use stimulation of no more than 25 volts and 0.2 amperes, although other scenarios may suffice and include the range progressing by 0.1 ampere intervals from 0.1 to 2.0 amperes.

In an embodiment, a stimulation algorithm (e.g., programmed in controller 122) is based on rhythmic timing of breathing while sleeping. During sleep breathing may slow to a pace of approximately one inhalation every 5 to 7 seconds. One embodiment of the simulation algorithm may be adjustable between multiple stimulations per second to one stimulation every 600 seconds. A setting may be once every 5 to 7 seconds such that the patient receives approximately one stimulation for each inhalation. The stimulation frequency may be adjusted up or down based on the severity, frequency, and duration of the hypoxia and apnea events.

In some embodiments stimulation is not based on biofeedback from sensors. However, in other embodiments stimulus can be based on biofeedback such as onset of respiration as detected via changes in thoracic impedance, a strain gauge strap worn across the chest, and the like. Such feedback may be coupled to controller 122 (e.g., radiofrequency (RF), direct interconnects). Stimulus may be provided only when respiration is not detected. However, in some embodiments continuous stimulation may be provided.

In an embodiment, electrodes 115 and/or electrode 125 are moveable. For example, electrode 115 may adhere to the inside of mask 110 via a hook and loop system. Thus, a user or medical practitioner may affix electrode 115 at various locations until proper stimulus at the lowest power level produces the desired effect on the target muscles and airway. With bilateral stimulation, the user may locate the left and right TMJ electrodes non-symmetrically (i.e., at different locations near the TMJ) to “tweak” stimulation to be most effective in light of anatomical concerns (e.g., scar tissue, acne, beard, variations in skin conduction).

In an embodiment of the invention, a collar is used to position electrodes on a user so current travelling between the electrodes can stimulate nerves that control the geometry of the collar user's airway (e.g., pharynx, neck, throat, mouth, trachea, and the like).

FIG. 2 includes a collar in an embodiment of the invention. Collar 200 includes first portion 210 that includes electrode 215, electrode 226, and module 220. Elements 215, 226, and 220 may be coupled to one another via interconnects (e.g., wires) 230, 235. Module 220 may include power source 221 (e.g., rechargeable battery), controller 222, user interface 123, and user input 124. Power source 221 may not necessarily include a battery but may instead couple to auxiliary power via an adaptor coupled to 110 volt power.

In an embodiment, electrode 225 (included in optional portion 237) may be substituted for electrode 226 to provide a stimulus vector similar to that of FIG. 1. Electrode 225 may couple to controller 220 via interconnect 236. In other embodiments, electrodes 225 and 226 may both be included in addition to electrode 215.

In an embodiment, collar 200 provides bilateral stimulation (but in other embodiments may provide for only unilateral stimulation). For example, FIG. 2 depicts electrode 215 near the patient's right TMJ along with electrode 226 near the throat and HGN 241. However, while not shown another electrode could be located near the patient's left TMJ. The left TMJ electrode could provide stimulation current to electrode 225 or to another electrode located near the chin and/or another electrode on the left next near the HGN. Also, the left TMJ electrode may receive power from module 220 or from another module located on the left side of collar 200. Also, controller 222 could be used to drive stimulus via the left TMJ or another controller could do so. As a result, stimulus to both the left and right TMJs may provide simultaneous stimulation to left and right nerve bundles located in the jaw and neck. These nerves control the muscles surrounding the airway. By stimulating these nerves the “airway muscles” are activated and the airway is kept open.

As with FIG. 1, collar 200 may stimulate (e.g., continuously or periodically) target nerves and/or muscles based on a programmed pulsing schedule delivered via, for example, controller 222. Target nerves for electrode 215 include peripheral nerves in the head and neck. Target muscles for electrode 215 stimulation include masseter muscle 240 and pharyngeal airway muscles such as geniohyoid, genioglossus, styloglossus, and hypoglossus muscles. As noted above, electrode 226 is near the throat and HGN 241.

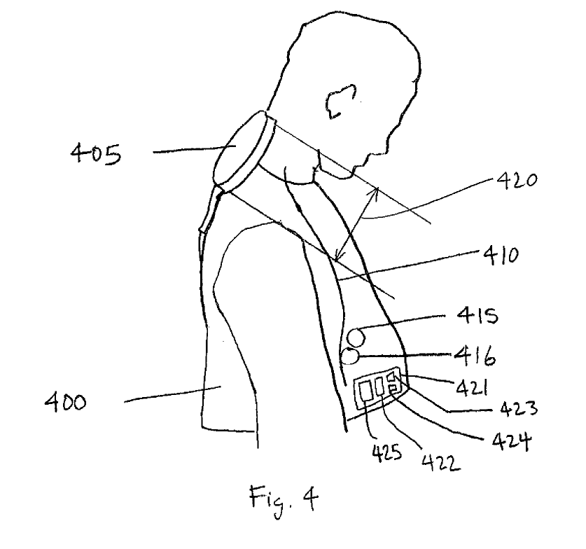

FIG. 4 includes vest 400 in an embodiment of the invention. A magnetic inductance source 405 is coupled to vest 400 using, for example, a pocket for magnetic source 405. Vest 400 may be worn during sleep (but other embodiments are suitable to worn while awake). Magnetic source 405 (e.g., magnetic and/or coil for electromagnetic induction (described more fully below)) stimulates phrenic nerve (PN) 410 via magnetic field 420. Source 405 may couple to a power source (e.g., battery or 110 volt supply). Electrodes 415, 416 may be used to detect apnea, which once sensed may be used to trigger stimulation from magnetic source 405. Electrodes may be directly included in vest 400 or indirectly coupled to vest 400 via cables and the like. Magnetic stimulation from source 405 may result in relatively fast nerve conduction time when compared to direct electrical lead stimulation conduction times. In an embodiment, magnetic field 420 originates adjacent the cervical spine and points toward the anterior exit of PN 410 (although may originate elsewhere, such as near the thoracic or lumbar spine, and directed elsewhere, such as superior or inferior to the anterior exit of PN410, in other embodiments). In an embodiment, magnetic source 405 may be located over PN 410, over the anterior thorax, below the clavicle, and approximately between the first and second ribs. In an embodiment, the magnetic field may be directed between the user's first and second ribs. Directing the field as illustrated results in stimulating PN 410 in a more distal location (i.e., closer to the diaphragm) than can be achieved with direct electrical stimulation of the diaphragm (e.g., when electrical stimulation is applied proximal to the neck) since the magnetic stimulation transverses the user to the diaphragm.

As with FIGS. 1 and 2, vest 400 may include module 421. Module 421 may include power source 425 (e.g., rechargeable battery), controller 422, user interface 423, and user input 424. Vest 400 may stimulate (e.g., continuously or periodically) target nerves (e.g., PN 410) and/or muscles based on a programmed pulsing schedule delivered via, for example, controller 422. Target muscles for stimulation include the diaphragm, stimulated based on stimulus of PN 410 via field 420.

In an embodiment, controller 422 determines when stimulation is needed and provides stimulation via programmed algorithms as described herein. Sensing may be performed based on biofeedback (e.g., onset of respiration as detected via changes in thoracic impedance, a strain gauge strap worn across the chest, and the like). Stimulus may be provided only when respiration is not detected.

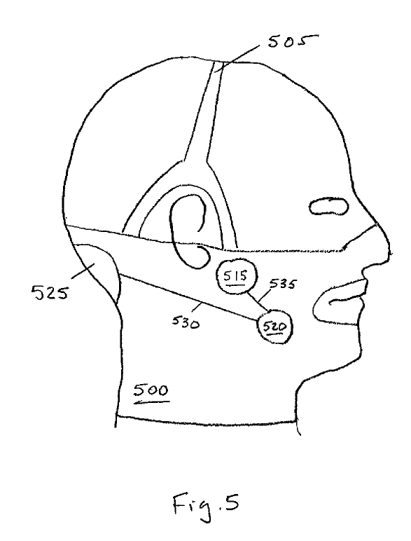

FIG. 5 includes a mask in an embodiment of the invention. Mask 500 includes electrode 515, electrode 525, and module 520. Elements 515, 525, and 520 may be coupled to one another via interconnects (e.g., wires) 530, 535. Module 520 may include the functionality and components previously discussed with FIG. 1, module 120. A difference from FIG. 1, however, is the location of electrodes 515, 525. Specifically, electrode 515 is still located near the TMJ or, in another embodiment, at or near the mastoid process. Electrode 525, however, is located at or near the inion. Thus, a stimulus vector between electrodes 515, 525 is now directed at the proximal HGN (whereas the distal portion of the HGN is more the focus in FIG. 1). Electrodes 515, 525 may be movable within mask 500 (e.g., electrodes may detachably attach to mask 500 via a hook and loop system) so one mask can provide for electrode embodiments seen in both FIGS. 1 and 5.

Embodiments of the invention may have various methods of use. For example, stimulus based on electrodes 115, 125 may open airway muscles as described above (e.g., vector 390 stimulates HGN and/or MN to open airway. However, depending on circumstances particular to a user (e.g., anatomy, severity of apnea, weight, obesity) such a user may find locating electrode 115 along the TMJ area and electrode 125 near the chin area may actually close or narrow the user airway. However, such a user may still use mask 100 to diminish apnea.

Stimulus based on electrodes 115, 125 may: (1) exhaust muscles whose over-activity results in apnea (resulting in those muscles being unable to activate as much) to thereby lessen apnea, (2) stimulate other nerves or muscles which may cause additional muscles to relax and thereby lessen apnea, or (3) stimulate other nerves or muscles which may cause additional muscles to activate and thereby lessen apnea. This lessening of apnea may be caused directly by activation of sensory nerves. However, the lessening may also be caused indirectly by activation of other muscles that lead to relaxation of the problematic muscles or activation of other muscles that may decrease apnea. The indirect activation may be due to excitation of afferent pathways.

In an embodiment, a user may use mask 100 while awake, such as, one hour before going to sleep. This activation may work based on any of the different modalities described above to reduce apnea. Mask 100 may affect both afferent and efferent nerves. During the one hour pre-sleep stimulation, mask 100 will stimulate the muscles under the chin and the distal lower tongue. This may condition the brain to place the tongue and throat in the proper position (while at the same time the brain is preparing for sleep). Therapy (e.g., over weeks or months) may appropriately recondition muscles to be in their proper position over the entire course of the sleep duration. The reconditioning may be based on a neurological response to the stimulation, which is biochemical nature. This release of chemicals prior to sleep may cause the brain to provide adequate neurological directions to keep the obstructions from occurring. For example, use of mask 100 may cause repeated hypoxic bouts in some individuals, which may lead to respiratory plasticity such as long term facilitation (LTF). This LTF may strengthen the ability of respiratory motoneurons to trigger contraction of breathing muscles. Thus the repeated hypoxic events (induced by mask 100) may trigger LTF of hypoglossal motoneuron activity and genioglossus muscle tone. In short, use of mask 100 may be a training tool for the brain to learn/remember how to breathe during sleep.

While noninvasive surface electrodes are shown in many of the above embodiments, with other embodiments implantable or percutaneous electrodes may be used. Such electrodes may receive power via electromagnetic induction.

Also, magnetic inductance can stimulate the same nerves/muscles described above. For example, a magnet inductance coil (or coils) can be placed under the chin for OSA treatment or at the TMJ for TMJ treatment (described more fully below). For example, electromagnetic stimulation (e.g., pulsed electromagnetic stimulation (“PES”)) passes electric current through a coil to generate an electromagnetic field, which induces a current within a conductive material (e.g., a nerve) placed inside the electromagnetic field. In other words, PES may stimulate a nerve positioned within the electromagnetic field to affect a muscle controlled by that nerve.

In an embodiment, mask 100 may contain one or more conductive coils under the chin. In other embodiments, mask 100 may contain one or more conductive coils at or near the TMJ. In other embodiments mask 100 may contain one or more conductive coils at or near the TMJ and at or near the chin. Any of these embodiments may produce a pulsed magnetic field that will flow across, for example, HGN 341 and/or MN342. The coils may take any of several known configurations (e.g., helical pattern, figure eight coil, four leaf clover coil, Helmholtz coil, modified Helmholtz coil, or a combination thereof).

Various embodiments (e.g., mask 100 or collar 200) may be useful as a treatment for TMJ disorders. For instance, many of the same muscle groups targeted in treating sleep apnea are the same muscles used in treating TMJ disorders. Specifically, electrode 115 may be the cathode and electrode 125 may be the anode. The resultant invoked nerve and/or muscle response may occur near the cathode at or near the TMJ. This may exercise muscles associated with the TMJ in a therapeutic manner.

Different embodiments may work together (e.g., using vest 400 in conjunction with mask 100). For example, vest 400 may be used as a treatment for CSA. The patient, however, may suffer from both CSA and OSA. Such a patient may use both vest 400 (for CSA therapy) and mask 100/collar 200 (for OSA therapy). Specifically, mask 100/collar 200 may couple to vest 400, which may include sensing modules to monitor and analyze the patient's breathing patterns (because therapy may only be supplied for CSA when the patient is actually experiencing apnea). The stimulation frequency may be dependent on vest 400 monitoring these breathing patterns and stimulating only when necessary. Vest 400 may invoke inspiration directly through electromagnetic stimulation of PN 410. However this effort may be ineffective if the patient is also concurrently suffering from OSA. Thus, simultaneous stimulation/therapy for both CSA and OSA may be used.

Thus, an embodiment includes a device, system, and method with a garment (e.g., mask or collar), which includes first and second electrodes both coupled to a power source and a controller, configured to locate the first electrode at a user's temporomandibular joint area and the second electrode at the user's chin area. Current is supplied between the first and second electrodes to stimulate the user's airway muscles (e.g., jaw, throat, tongue) and open the user's airway and limit sleep apnea. The process for limit sleep apnea may be conducted via various level of directness. For example, apnea may be limited by relaxing an additional muscle (e.g., one other than muscle being directly stimulated by the device) based on supplying the current between the first and second electrodes; and then opening the user's airway based on relaxing the additional muscle. As another example, apnea may be limited by inducing respiratory LTF based on stimulating the user's airway muscles; and then opening the user's airway based on the LTF. The time between stimulating muscles/nerves with the garment and actually seeing therapeutic results may not be immediate but may have an delayed onset of minutes, hours, days, or weeks (e.g., based on training). Various nerves (e.g., HGN, MN) may be stimulated. Stimulus may be applied during sleep, while awake, or both.

In an embodiment, a garment may include a third electrode at the user's neck area. Apnea may be limited by supplying current to the third electrode so as to simultaneously stimulate jaw and pharyngeal airway muscles based on simultaneously supplying current to third electrode and current between the first and second electrodes.

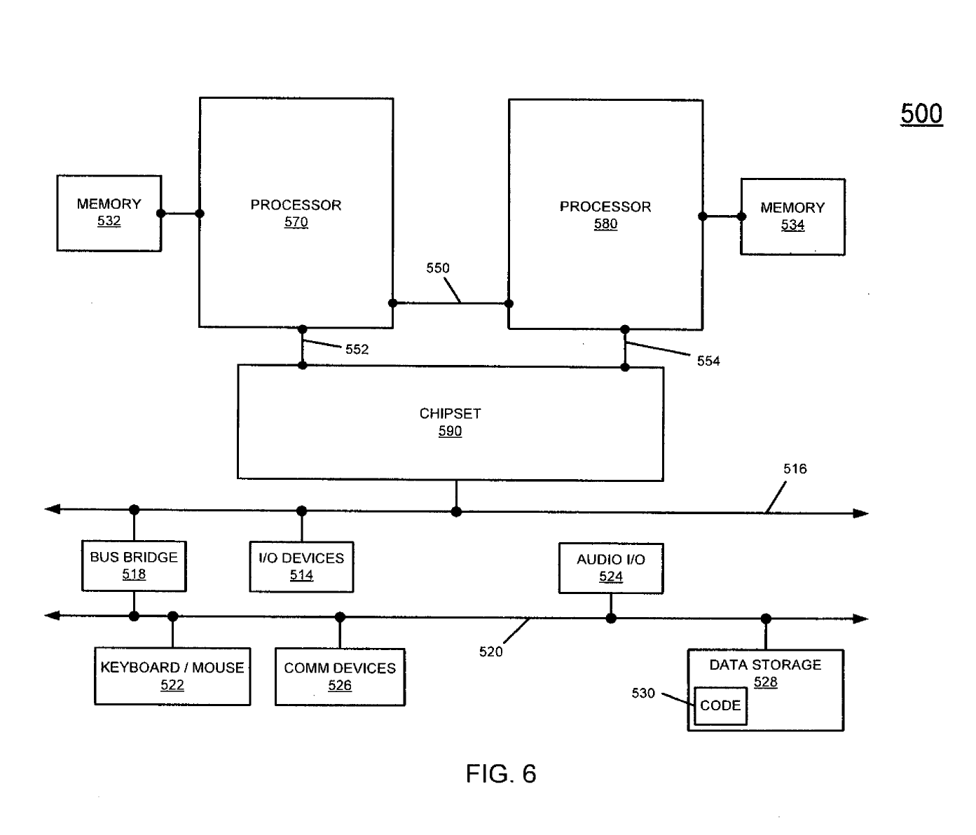

Embodiments may be implemented in many different system types. Referring to FIG. 6, shown is a block diagram of a system in accordance with an embodiment of the present invention. Controller 122 (FIG. 1) may interact with system 500 (FIG. 6). For example, controller 122 may be programmed via interfacing (e.g., RF, magnetic, direct connection) with system 500. Portions of system 500 may be duplicated or located within module 220. Controller 122 may interface an electrical stimulation sub system (and/or magnetic stimulation sub system) included within module 120. Controller 122 may send instructions to determine pacing or stimulation protocols. For example, a protocol may include various criteria such as Wave Form (e.g., biphase square pulse), Pulse Rate (e.g., adjustable from 0.5-150 Hz, Pulse Width (e.g., 50-300 microseconds), Output Voltage (e.g., 0 to 50 V and Load of 1000 ohm), Output Intensity (e.g., adjustable, 0-105 mA).

Multiprocessor system 500 (e.g., smart phone, laptop, netbook, personal computer, user wearable module, etc.) is a point-to-point interconnect system, and includes a first processor 570 and a second processor 580 coupled via a point-to-point interconnect 550. Each of processors 570 and 580 may be multicore processors. The term “processor” may refer to any device or portion of a device that processes electronic data from registers and/or memory to transform that electronic data into other electronic data that may be stored in registers and/or memory.

First processor 570 may include a memory controller hub (MCH) and point-to-point (P-P) interfaces. Similarly, second processor 580 may include a MCH and P-P interfaces. The MCHs may couple the processors to respective memories, namely memory 532 and memory 534, which may be portions of main memory (e.g., a dynamic random access memory (DRAM)) locally attached to the respective processors. First processor 570 and second processor 580 may be coupled to a chipset 590 via P-P interconnects, respectively. Chipset 590 may include P-P interfaces.

Furthermore, chipset 590 may be coupled to a first bus 516 via an interface. Various input/output (I/O) devices 514 may be coupled to first bus 516, along with a bus bridge 518, which couples first bus 516 to a second bus 520. Various devices may be coupled to second bus 520 including, for example, a keyboard/mouse 522, communication devices 526, and data storage unit 528 such as a disk drive or other mass storage device, which may include code 530, in one embodiment. Further, an audio I/O 524 may be coupled to second bus 520.

Embodiments may be implemented in code and may be stored on a storage medium having stored thereon instructions which can be used to program a system to perform the instructions. The storage medium may include, but is not limited to, any type of disk including floppy disks, optical disks, optical disks, solid state drives (SSDs), compact disk read-only memories (CD-ROMs), compact disk rewritables (CD-RWs), and magneto-optical disks, semiconductor devices such as read-only memories (ROMs), random access memories (RAMs) such as dynamic random access memories (DRAMs), static random access memories (SRAMs), erasable programmable read-only memories (EPROMs), flash memories, electrically erasable programmable read-only memories (EEPROMs), magnetic or optical cards, or any other type of media suitable for storing electronic instructions.

Embodiments of the invention may be described herein with reference to data such as instructions, functions, procedures, data structures, application programs, configuration settings, code, and the like. When the data is accessed by a machine, the machine may respond by performing tasks, defining abstract data types, establishing low-level hardware contexts, and/or performing other operations, as described in greater detail herein. The data may be stored in volatile and/or non-volatile data storage. For purposes of this disclosure, the terms “code” or “program” cover a broad range of components and constructs, including applications, drivers, processes, routines, methods, modules, and subprograms. Thus, the terms “code” or “program” may be used to refer to any collection of instructions which, when executed by a processing system, performs a desired operation or operations. In addition, alternative embodiments may include processes that use fewer than all of the disclosed operations, processes that use additional operations, processes that use the same operations in a different sequence, and processes in which the individual operations disclosed herein are combined, subdivided, or otherwise altered.

As used herein a processor or controller may include control logic intended to represent any of a wide variety of control logic known in the art and, as such, may well be implemented as a microprocessor, a micro-controller, a field-programmable gate array (FPGA), application specific integrated circuit (ASIC), programmable logic device (PLD) and the like. In some implementations, controller 122, 222 and the like are intended to represent content (e.g., software instructions, etc.), which when executed implements the features (e.g., sensing and pacing features) described herein.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A method comprising:
providing a garment, which includes first and second electrodes both coupled to a power source and a controller, configured to locate the first electrode at a user's temporomandibular joint area and the second electrode at the user's chin area;
wearing the garment to noninvasively locate the first electrode at the user's temporomandibular joint area and the second electrode at the user's chin area;
supplying a current between the first and second electrodes;
stimulating one or more of the user's airway muscles based on supplying the current between the first and second electrodes; and
opening the user's airway, based on the stimulus, to limit sleep apnea.

2. The method of claim 1 including stimulating the user's hypoglossal nerve based on supplying the current between the first and second electrodes.

3. The method of claim 1 including stimulating, based on supplying the current between the first and second electrodes, one of the user's masseter muscle and masseteric nerve.

4. The method of claim 3 including:
providing the garment, which includes a third electrode coupled to the power source and the controller, configured to locate the third electrode at the user's neck area;
wearing the garment to noninvasively locate the third electrode at the user's neck area;
supplying additional current to the third electrode;
simultaneously stimulating one of the one or more of the user's airway muscles based on simultaneously supplying the additional current to third electrode and supplying the current between the first and second electrodes; and
opening the user's airway, based on the current and the additional current, to limit sleep apnea.

5. The method of claim 1 including:
sleeping; and
while sleeping, (a) supplying the current between the first and second electrodes;
(b) stimulating the one or more of the user's airway muscles based on supplying the current between the first and second electrodes; and (c) opening the user's airway, based on the stimulus, to limit sleep apnea.

6. The method of claim 1 including:
remaining awake; and
while remaining awake, (a) supplying the current between the first and second electrodes; (b) stimulating the one or more of the user's airway muscles based on supplying the current between the first and second electrodes; and (c) opening the user's airway, based on the stimulus, to limit sleep apnea.

7. The method of claim 1, wherein:
supplying the current between the first and second electrodes includes supplying the current at a level less than 0.3 amperes and at a frequency between 1 pulse every 4 to 8 seconds;
the first and second electrodes are located between 4 and 6 inches from each other; and
the one or more of the user's airway muscles include at least one of jaw and pharyngeal muscles.

8. The method of claim 1, wherein the garment (a) includes one of a mask and a collar, and (b) is configured to locate the first electrode directly over the user's temporomandibular joint and the second electrode directly under the user's chin.

9. The method of claim 1, including:
relaxing an additional muscle based on supplying the current between the first and second electrodes; and
opening the user's airway based on relaxing the additional muscle.

10. The method of claim 1, including:
inducing respiratory long term facilitation (LTF) based on stimulating the user's airway muscles; and
opening the user's airway based on the LTF.

11. A system comprising:
first and second electrodes and a controller; and
a garment to include the first and second electrodes, a power source, and the controller, the first and second electrodes both to couple to the power source and the controller;
wherein the garment, when worn and operated, is configured to: (a) locate the first electrode at a user's temporomandibular joint area and the second electrode at the user's chin area, (b) supply a current between the first and second electrodes; (c) stimulate one or more of the user's airway muscles based on supplying the current between the first and second electrodes; and (d) open the user's airway, based on the stimulus, to limit sleep apnea.

12. The apparatus of claim 11, wherein the garment is configured to locate the first electrode and the second electrode so as to stimulate the user's hypoglossal nerve based on supplying the current between the first and second electrodes.

13. The apparatus of claim 11, wherein the garment is configured to locate the first electrode and the second electrode so as to stimulate one of the user's masseter muscle and masseteric nerve.

14. The apparatus of claim 11 including:
a third electrode to couple to the power source and the controller;
wherein the garment is configured to: (a) locate the third electrode at the user's neck area; (b) supply additional current to the third electrode; (c) simultaneously stimulate one of the one or more of the user's airway muscles based on simultaneously supplying the additional current to the third electrode and supplying the current between the first and second electrodes; and (d) open the user's airway, based on the current and the additional current, to limit sleep apnea.

15. The apparatus claim 11, wherein the garment is configured to (a) supply the current between the first and second electrodes at a level less than 0.3 amperes and at a frequency between 1 pulse every 4 to 8 seconds; (b) locate the first and second electrodes between 4 and 6 inches from each other; and (c) the one or more of the user's airway muscles include at least one of jaw and pharyngeal muscles.

16. The apparatus claim 11, wherein the garment (a) includes one of a mask and a collar, and (b) is configured to locate the first electrode directly over the user's temporomandibular joint and the second electrode directly under the user's chin.

17. A method comprising:
providing a garment, which includes a magnetic inductance source coupled to a power source and a controller, configured to noninvasively locate the magnetic inductance source adjacent a user's cervical spine;
providing a first sensor to detect cessation of breathing, the first sensor coupled to the controller;
wearing the garment to noninvasively locate the magnetic inductance source adjacent the user's cervical spine;
coupling the first sensor to the user and detecting the cessation of breathing based on the first sensor;
producing a magnetic field, via the magnetic inductance source, based on detecting the cessation of breathing;
directing the magnetic field towards the anterior exit of the user's phrenic nerve;
magnetically inducing, via the magnetic field, stimulation of the user's phrenic nerve; and
stimulating the user's diaphragm, based on stimulating the user's phrenic nerve, to limit sleep apnea.

18. The method of claim 17, wherein the magnetic inductance source includes one of a coil and a magnet.

19. The method of claim 17, wherein directing the magnetic field towards the anterior exit of the user's phrenic nerve includes directing the magnetic field between the user's first and second ribs.
